(19) United States
(12) Patent Application Publication
(10) Pub. No.: US 2026/0031234 A1
(43) Pub. Date: Jan. 29, 2026

(54) Systems and Methods for Predicting Outcomes Using Large Language Models

(71) Applicant: Elevance Health, Inc., Indianapolis, IN (US)

(72) Inventors: Shaheen GAUHER, Sharon, MA (US); Teja Simha KANCHINADAM, Charlotte, NC (US)

(21) Appl. No.: 19/282,259

(22) Filed: Jul. 28, 2025

Related U.S. Application Data
(60) Provisional application No. 63/675,987, filed on Jul. 26, 2024.

(51) Int. Cl. G16H 50/30; G06F 40/284; G06N 20/00

(52) U.S. Cl. CPC G16H 50/30; G06F 40/284; G06N 20/00

(57) ABSTRACT

Systems, methods and user interfaces are provided for training a causal language model for predicting outcomes using large language models. The method may include obtaining a training dataset that includes structured data including codes. The method may also include preprocessing the structured data to convert raw events data into a structured token sequence. The method may also include training a causal language model using the structured token sequence to predict an outcome. The method may also include generating a synthetic dataset based on fine-tuning the trained causal language model on an evaluation dataset. The method may also include evaluating the trained causal language model.

### CROSS-REFERENCE TO RELATED APPLICATIONS

This application claims priority to and the benefit of U.S. Provisional Application No. 63/675,987, filed Jul. 26, 2024, the entirety of which is incorporated herein by reference.

### BACKGROUND

Administrative claims data is an important component of the healthcare sector. This data adeptly captures the intricacies of the practice of medicine. Claims data provides extensive coverage, capturing detailed patient histories through insurance reimbursement records. Claims data is rich in diagnostic and procedural information encoded in medical codes like International Classification of Diseases, Tenth Revision (ICD-10-CM) and Current Procedural Terminology (CPT). Claims data is pivotal in understanding healthcare delivery and patientcare patterns. However, complexity of the claims data challenges traditional data processing, necessitating innovative artificial intelligence (AI) approaches.

The emergence of large language models (LLMs) signifies a transformative phase in data analytics, particularly within the healthcare sector, where their ability to process vast, unstructured datasets has groundbreaking potential. While language models like BioBERT, SCIBERT, Pub-MedBERT, and ClinicalBERT have excelled in biomedical NLP tasks, and conversational models, such as Med-PaLM, Med-PaLM 2, ChatDoctor, and Baize-health have shown impressive results in medical questionnaires, these models exhibit limitations in fully grasping the practice of medicine and predicting clinical outcomes. These models, despite their advancements, often lack the depth of understanding needed to accurately predict patient-specific clinical outcomes, a key aspect in the realm of medical practice and decision-making support. Similar problems exist in industries outside of the medical industry. LLMs similarly struggle to predict outcomes in sectors, which use codes to identify events and/or event requests.

### SUMMARY

Accordingly, there is a need for systems, methods and interfaces that predict outcomes using large language models. Healthcare tasks, such as predicting clinical outcomes across medical and surgical populations, disease prediction, predicting patient health journeys, may be approached with supervised learning on task-specific datasets. According to the techniques described herein, language models may begin to learn these tasks without any explicit supervision when trained on a new dataset of billions of administrative event requests (e.g., claims), which essentially encapsulates the practice of the industry/sector (e.g., medicine), offering a unique perspective on care (e.g., patient care) and treatment patterns. An example model MediClaimGPT is described herein. The model, which may include a 125 million parameter transformer, demonstrates strong zero-shot predictive capabilities, forecasting events (e.g., patient health events) across four evaluation datasets, with its capabilities further demonstrated in various downstream tasks. A significant application of MediClaimGPT may be in generating high quality, synthetic events data (e.g., clinically plausible synthetic claims data), enhancing data utility (e.g., healthcare data utility) while preserving privacy (e.g., patient privacy). In this way, language models may be trained and/or used to handle complex datasets in healthcare and related fields.

In one aspect, a method is provided for predicting outcomes using large language models, according to some embodiments. The method may include obtaining a training dataset that includes structured data including codes. The method may also include preprocessing the structured data to convert raw event requests into a structured token sequence. The method may also include training a causal language model using the structured token sequence to predict an outcome.

In some embodiments, the structured data includes a respective dataset for a plurality of individuals. Each dataset may include a plurality of event requests. Each individual may have a corresponding set of event requests. Each event request may include a set of codes. Each code may be either a diagnosis code, a procedural code, a drug code, a lab code or any other type of medical code or an encapsulation of a medical code, such as Clinical Classifications Software (CCS) system.

Each event request in the structured data may correspond to an individual-provider encounter. Each event request may aggregate medical codes (e.g., diagnosis codes, procedure codes) in a non-sequential order.

In some embodiments, preprocessing the structured data includes performing a sorting algorithm to organize codes within each event request in the structured data into a clinically logical sequence, including chronologically ordering event requests for a respective individual to form a temporally sequenced dataset thereby enabling a machine learning model to learn chronological order of events.

In some embodiments, the sorting algorithm σ organizes the codes within each event request cᵢⱼ into a clinically logical sequence,

$${c}_{ij}^{l} = σ ( {e}_{ijl} , {e}_{ij 2} , … , {e}_{ijk} ) .$$

Event requests

$${C}_{i}^{l} = {c}_{i 1}^{l} , {c}_{i 2}^{l} , … , {c}_{iC}^{l}$$

may be chronologically ordered as

$${′}^{} = {⋃}_{p = 1}^{p} \{ sort ( {C}_{p} , date ) \}$$

forming a temporally sequenced dataset, enabling the causal language model to learn the chronological order of events.

In some embodiments, preprocessing the structured data includes intra-event request codes, inter-event request codes for the respective individual, and data for different individuals, thereby enabling batch data processing.

In some embodiments, preprocessing the structured data includes tokenizing the structured data using a tokenizer to obtain a sequence of tokens. The tokenizer may preserve one or more delimiter tokens to maintain context of data. The tokenizer may be trained on event requests data with a predetermined vocabulary size.

In some embodiments, the tokenizer uses Byte-Level Byte-Pair Encoding for creating a fixed-size vocabulary balancing medical language specificity with capacity of the causal language model.

In some embodiments, the causal language model is trained on the sequence of tokens to predict a subsequent token in the sequence, with a loss function measuring the accuracy of predictions

$$Loss ( Θ ) = - {\sum }_{t = 1}^{L} log P ( t \vert t - 1 , t - 2 , … , 1 ; Θ ) \cdot P ( t \vert t - 1 , t - 2 , … , 1 ; Θ )$$

represents the causal language model's assigned probability to a true next token t, given all previous tokens in the sequence.

In some embodiments, the training dataset includes event requests data that covers a plurality of individual demographics and conditions from a plurality of care settings.

In some embodiments, the training dataset includes billions of event requests corresponding to millions of individuals, tens of thousands of medical codes (e.g., diagnosis codes, procedure codes) and tens of thousands of unique procedure codes, and wherein the training dataset excludes invalid codes resulting from intake or ingestion errors.

In some embodiments, training the causal language model using the structured token sequence includes predicting a next code in the structured token sequence based on prior codes, thereby generating a sequence of codes for each claim in a causally coherent manner.

In some embodiments, predicting the next code is modeled as a probability distribution over possible codes.

In some embodiments, the probability distribution over the possible codes is formulated as P(eᵢⱼₖ|eᵢⱼ;Θ)=M(eᵢⱼ), wherein θ denotes the parameters of the causal language model, wherein sequence of codes eᵢⱼ=(eᵢⱼ₁, eᵢⱼ₂) . . . , eᵢⱼ₍ₖ₋₁₎) for the j^{th} event request of the i^{th} individual. The language model may predict the next code eᵢⱼₖ thereby generating the sequence of codes for each event request in a causally coherent manner, reflective of the actual progression of events documented in the event requests data.

In some embodiments, the causal language model includes a 12-layer transformer with 768-dimensional states across 12 attention heads, totaling about 125 million parameters.

In some embodiments, the causal language model is trained on a predetermined context size (e.g., 1,024-token context size) to capture detailed individual histories, using a predetermined batch size (e.g., a batch size of 512).

In some embodiments, the causal language model has a predetermined vocabulary size (e.g., a vocabulary size of 2,048) thereby optimizing handling of code hierarchies while maintaining computational efficiency.

In some embodiments, the method further includes using zero-shot prompting for forecasting outcomes.

In some embodiments, using zero-shot prompting includes inputting, to the causal language model, an individual's event request history for an observation period and analyzing output generated by the causal language model for event occurrence.

In some embodiments, temperature of the causal language model is set to 0.7, thereby balancing creativity and precision in generated outcomes.

In some embodiments, a maximum token size of 500 and top-k sampling with k=100 are used.

In some embodiments, the method further includes generating a synthetic dataset based on fine-tuning the trained causal language model on an evaluation dataset.

In some embodiments, fine-tuning the trained causal language model includes introducing special tokens |pos| and |neg| to enable the fine-tuned model to generate synthetic event requests corresponding to positive and negative samples, respectively

$${M}_{ft} = FineTune ( M , {eval}_{} , \vert pos \vert , \vert neg \vert )$$

wherein M denotes the trained causal language model, [symbol] ₑᵥₐₗ denotes the evaluation dataset, M_{ft} denotes the model after fine-tuning, and |pos| or |neg| are used prompts for generating the synthetic dataset.

In some embodiments, hyperparameter settings from training the causal language model are retained during the fine-tuning with the addition of a dropout rate of 0.5 and a learning rate of 6e-5, to fine-tune within 5 epochs. The fine-tuning may use a learning rate decay schedule with a warmup over 0.5% of training duration.

In another aspect, a computer system includes one or more processors, memory, and one or more programs stored in the memory. The programs are configured for execution by the one or more processors. The programs include instructions for performing any of the methods described herein.

In another aspect, a non-transitory computer readable storage medium stores one or more programs configured for execution by one or more processors of a computer system. The programs include instructions for performing any of the methods described herein.

### BRIEF DESCRIPTION OF THE DRAWINGS

FIG. 1 is a schematic diagram of claims data, according to some embodiments.

FIG. 2 shows a table of example prompts and corresponding responses from a model, according to some embodiments.

FIG. 3 shows an example of structured data for two individuals, according to some embodiments.

FIG. 4 shows a table for evaluation of the model in zero-shot prediction for different datasets, according to some embodiments.

FIG. 5 shows a table for classification performance across different representations and models for downstream prediction tasks, according to some embodiments.

FIG. 6 shows a table with results of evaluation of synthetic data, according to some embodiments.

FIG. 7 shows a graph plot 700 for topic diversity between real and synthetic claims for Spinal Fusion dataset, according to some embodiments.

FIG. 8 is a system diagram of an example outcome prediction system, according to some embodiments.

FIG. 9 is a flowchart of a method for predicting outcomes using large language models, according to some embodiments.

Like reference numerals refer to corresponding parts throughout the drawings.

### DESCRIPTION OF IMPLEMENTATIONS

Reference will now be made to various implementations, examples of which are illustrated in the accompanying drawings. In the following detailed description, numerous specific details are set forth in order to provide a thorough understanding of the invention and the described implementations. However, the invention may be practiced without these specific details. In other instances, well-known methods, procedures, components, and circuits have not been described in detail so as not to unnecessarily obscure aspects of the implementations.

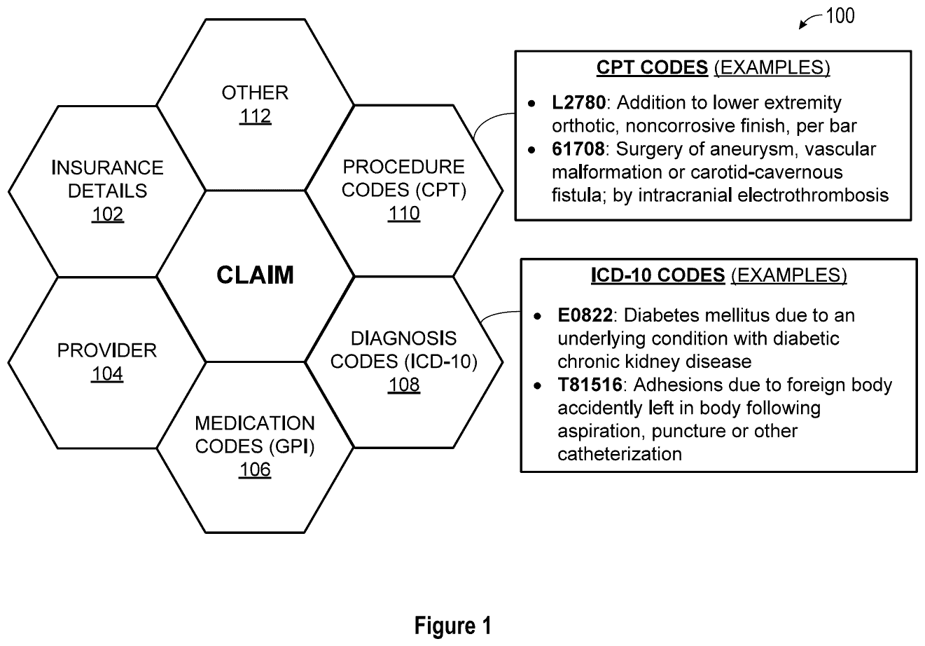

Disclosed embodiments enable prediction of outcomes (e.g., clinical outcomes) using large language models. Systems, methods and devices implementing the techniques in accordance with some embodiments are illustrated in FIGS. 1-7. Large Language Models (LLMs) may be used to manage and/or process complex data (e.g., healthcare data). Some embodiments structure administrative data (e.g., claims data) into a format suitable for LLMs. Zero-shot prompting may be used with LLMs for forecasting outcomes (e.g., patient health outcomes). Some embodiments train and/or use LLMs to produce realistic synthetic data while preserving privacy (e.g., patient privacy).

### Example Administrative Data

An event request, such as a claim, may be a bill submitted by providers (e.g., healthcare providers) to an insurance provider (e.g., patient's health insurance provider). Since by nature, event requests are transactional in nature, every encounter in a provider's office (e.g., a patient encounter in a physician's office, hospital, or other healthcare facility), may be captured in event request data (e.g., claims data) with rich details (e.g., details about diagnosis made, medications prescribed, procedures performed, and/or services availed) in the form of preestablished codes. Event requests data may follow a relatively consistent format and use a standard set of rules for coding (e.g., medical coding). Claims data may be a source of standardized patient information. FIG. 1 is a schematic diagram of claims data 100, according to some embodiments. Claims data may include insurance data 102, provider data 104, medication codes 106, diagnosis codes 108, procedure codes 110, and other administrative claims data 112.

### Example Codes

Codes, such as medical codes, may include diagnosis and procedure codes. Medical codes may be contained within a claim (sometimes referred to as an event request).
Diagnosis codes 108: Patient diagnosis, for example, may be captured in the form of International Classification of Diseases, Tenth Revision (ICD-10-CM) codes. These codes are preestablished and are used by providers (e.g., physicians and other healthcare providers in United States) to classify and code all diagnoses. These may be three to seven characters long where:
   The first three characters categorize the injury.
   The fourth through sixth characters describe in greater detail the cause, anatomical location and severity of an injury or illness.
   The seventh character is an extension digit and used to classify an initial, subsequent or sequela (late effect) treatment encounter.
Procedure codes 110: The services rendered for an individual (e.g., a patient) may be captured in the form of Current Procedural Terminology (CPT) codes. These codes may be designed to communicate uniform information about procedures (e.g., medical procedures among physicians, patients and other healthcare providers). CPT codes are broadly categorized into three main categories where each category is further divided to various levels typically defined by a range. For example, (80000 . . . 89398) are a set of codes for pathology and laboratory procedures.

While each code (e.g., medical codes) may have an associated English description, some embodiments use only the codes themselves. Converting codes in the claims to descriptions often disrupts textual coherence, leading to disjointed sentences and a lack of semantic flow. Moreover, using descriptions significantly increases the context length. For instance, converting a year of an individual's history (e.g., a patient's health history) into descriptions may result in an average sequence length of a large number of tokens (e.g., 32,000 tokens) using the tiktoken library. Considering that clinical event prediction typically requires more than two years of data, the sequence length becomes impractically long. Additionally, in zero-shot settings where the model may predict outcomes from an individual's history (e.g., predict clinical outcomes from a patient's history), using descriptions complicates the process, as generated text would require mapping back to codes for any operational use. This requirement could lead to new challenges in automated medical coding if the descriptions vary even slightly from standard codes.

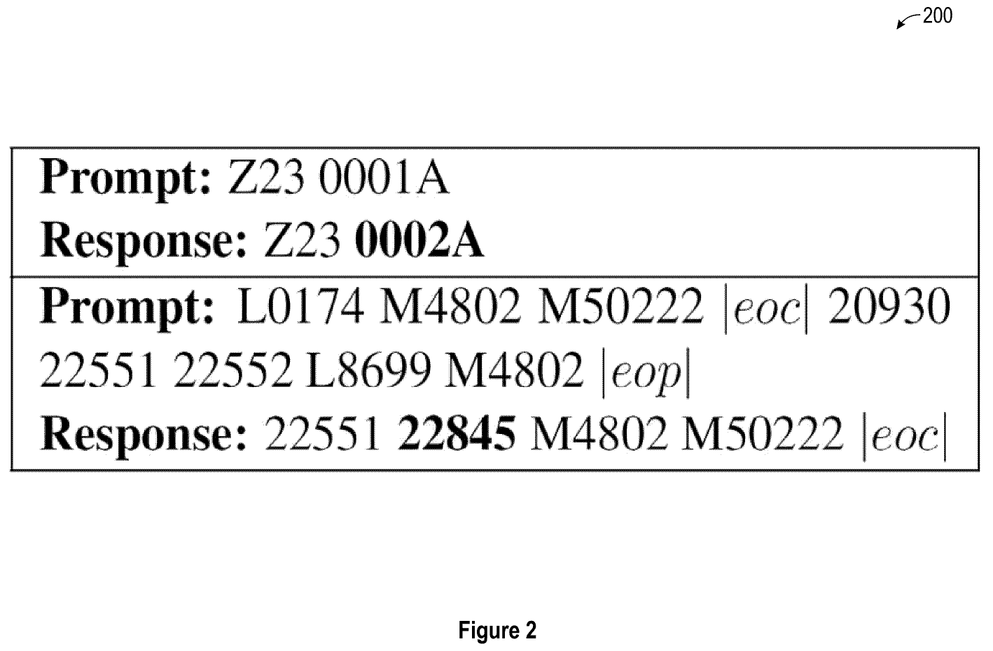

FIG. 2 shows a table 200 of example prompts and corresponding responses from MediClaimGPT (a model described herein), according to some embodiments. MediClaimGPT interprets medical codes. Medical codes are used for illustration, any similar code may be used. The first row or prompt illustrates vaccine sequence prediction (COVID-19 vaccine dosages) and the second row or response demonstrates surgical likelihood assessment for spinal conditions. These examples highlight MediClaimGPT's capacity in zero-shot settings to generate clinically relevant predictions.

Some embodiments perform causal language modeling using event requests data (e.g., healthcare claims data). These techniques may be used to capture the temporal and sequential nature of events (e.g., medical events) as reflected in claims data.

$$= {{⋃}_{p = 1}}^{P} \{ {{{⋃}_{c = 1}}^{C}}_{} \{ {e}_{1} , {e}_{2} , … , {e}_{\vert E \vert} \} \} \qquad ( 1 )$$

A dataset D may include P individuals (e.g., patients), each may be associated with a collection of C event requests (e.g., claims). For each individual pt, where i∈{1, . . . , P}, there may be a series of event requests cᵢ₁, cᵢ₂ . . . , c_{ic}. Each event request cᵢⱼ, with j∈{1, . . . , C}, may include a set of codes {eᵢⱼ, eᵢⱼ2, . . . , eᵢⱼₖ}, where each code eᵢⱼₖ may be either a diagnosis code (e.g., ICD-10-CM) or a procedural code (e.g., CPT).

In some embodiments, the task may be to utilize a causal language model M to predict the next code in the sequence given the prior codes. For a given sequence of codes eᵢⱼ= (eᵢⱼ₁, eᵢⱼ₂, . . . , eᵢⱼ₍ₖ₋₁₎) for the j^{th} event request of the i^{th} individual, a model may predict the next code eᵢⱼₖ. The prediction of the next code may be modeled as a probability distribution over the possible codes, formulated as:

$$P ( {e}_{ijk} \vert {e}_{ij} ; Θ ) = M ( {e}_{ij} ) \qquad ( 2 )$$

where θ denotes the parameters of the language model. The model's task across the dataset D may be to sequentially predict the next event code eᵢⱼₖ (e.g., a medical code), thereby generating the sequence of codes for each event request in a causally coherent manner, reflective of the actual progression of events (e.g., medical events) documented in the event request (e.g., claims) data.

### Example Data Processing/Pre-Processing

The preprocessing may include converting raw event requests (e.g., raw claims) into structured token sequences. Each event request (e.g., a claim, a record of patient-provider encounters), may aggregate diagnosis and procedure codes in a non-sequential order. To align these for language modeling, a sorting algorithm σ may organize the codes within each event request cᵢⱼ into a clinically logical sequence,

$${c}_{ij}^{l} = σ ( {e}_{ijl} , {e}_{ij 2} , … , {e}_{ijk} ) .$$

Futhermore, event requests (e.g., patient claims)

$${c}_{i}^{l} = {c}_{i 1}^{l} , {c}_{i 2}^{l} , … , {c}_{iC}^{l}$$

may be chronologically ordered as

$${′}^{} = {⋃}_{p = 1}^{P} \{ sort ( {C}_{p} , date ) \} \qquad ( 3 )$$

forming a temporally sequenced dataset, enabling the model to learn the chronological order of events (e.g., medical events).

### Example Utilization of Special Tokens

Specialized delimiter tokens may be employed at various levels within the events requests data (e.g., claims data) to enhance the causal language model's understanding of its structure. For example, for claims data, intra-claim codes may be concatenated with a white space character in their sorted order, represented as

$${c}_{ij}^{*} = {e}_{ij 1}^{l} {e}_{ij 2}^{l} … {e}_{ijk}^{l} .$$

For inter-claim concatenation, claims of a patient may be combined using a unique delimiter |eoc|, denoting each claim as a distinct entity, expressed as

$${p}_{i}^{*} = {c}_{i 1}^{*} \vert eoc \vert {c}_{i 2}^{*} \vert eoc \vert … \vert eoc \vert {c}_{iC}^{*} .$$

Similarly, interpatient data may be differentiated using |eop|, critical for batched data processing, formalized as

$${D}^{*} = {p}_{1}^{*} \vert eop \vert {p}_{2}^{*} \vert eop \vert … \vert eop \vert {p}_{P}^{*} .$$

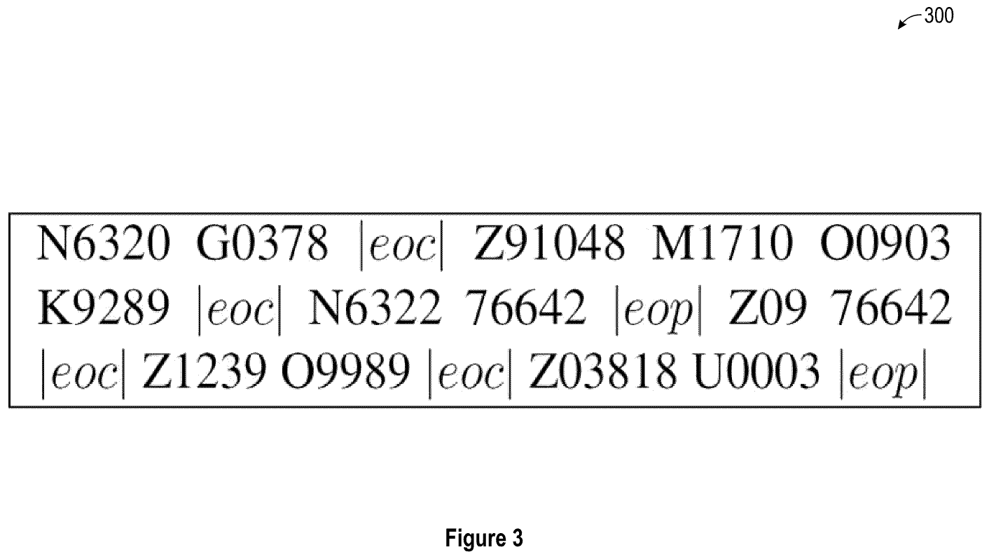

FIG. 3 shows an example of structured data 300 for two individuals, according to some embodiments.

### Example Tokenization and Training

Some embodiments use a tokenizer. This tokenizer may be trained on the event requests data D* (e.g., claims data) with a vocabulary size of V. The special tokens described above remain unchanged by the tokenizer, as these tokens may serve as delimiters in the data and may be preserved in their original form to maintain context of the medical data. The tokenization may utilize Byte-Level Byte Pair Encoding (BPE), creating a fixed-size vocabulary and thereby, balancing language specificity for the particular field (e.g., medical language for the medical field) with the model's capacity.

The learned tokenizer may be applied to dataset D*, resulting in a sequence of tokens. The causal language model M may be trained on these sequences to predict the correct subsequent token in a sequence, with a loss function, typically cross-entropy, measuring the accuracy of predictions

$$Loss ( Θ ) = - {\sum }_{t = 1}^{L} log P ( t \vert t - 1 , t - 2 , … , 1 ; Θ ) \qquad ( 4 )$$

where P(t|t−1,t−2, . . . , 1; 0) represents the model's assigned probability to the true next token t, given all previous tokens in the sequence.

### Example Experiments

### Example Pre-training

In some embodiments, MediClaimGPT architecture is similar to the OpenAI's GPT-2, may feature a 12-layer transformer with 768-dimensional states across 12 attention heads, totaling about 125 million parameters. The model may be trained on a 1024-token context size to capture detailed individual histories (e.g., patient histories), it may use a batch size of 512. Its vocabulary size of 2048 may optimize the handling of medical code hierarchies while maintaining computational efficiency. In experiments, MediClaimGPT model demonstrated a token-level perplexity of 1.02 on the validation dataset, indicating high predictive accuracy.

### Example Evaluation Setup

MediClaimGPT was evaluated in the following key areas:
Zero-shot prediction: to assess zero-shot prediction capabilities for clinical outcomes using patient health history, without modifying the model's weights.
Downstream prediction: to assess the model's performance in downstream clinical classification tasks.
Synthetic data generation: to validate the model's ability in generating clinically plausible synthetic data while ensuring privacy.

The example study examined four clinical cohorts, each focused on predicting a specific clinical event, thereby forming our evaluation datasets Deval. These datasets included: 1) Spinal fusion surgery (11,000 patients), 2) Knee replacement (54,000 patients), 3) Hip replacement (24,000 patients), and 4) Endoscopy (251,000 patients). These datasets were curated with the help of clinical experts and each dataset included patient claims from a two-year observation window, with a binary target indicating whether the clinical event occurs in a subsequent six-month prediction window. These events were selected for their potential for therapeutic prevention and significant cost implications. A clinical event may be identified by specific procedures or diagnoses, such as codes (22532, 22533, etc.) for spinal fusion surgery. In zero-shot settings, patient claims from the observation period may serve as input for MediClaimGPT, with its output analyzed to assess the occurrence of clinical events. For downstream prediction tasks, these claims may train a classifier using binary targets. The methodology for synthetic data generation may include fine-tuning on these claims, as described below, according to some embodiments.

### Example Zero-Shot Prediction

To evaluate MediClaimGPT in zero-shot settings, the patient's claim history from the observation period (input) may be provided to the model as ‘prompt,’ and the generated output may be later analyzed for clinical event occurrence. For example, if the output contained any of the code from a predetermined set (e.g., 22532, 22533), the patient may be likely to have a spinal fusion surgery in the future. This approach is particularly valuable as it leverages the model as-is, without changing the weights of the model or even downstream modeling. More details on experimental setup are described below, according to some embodiments.

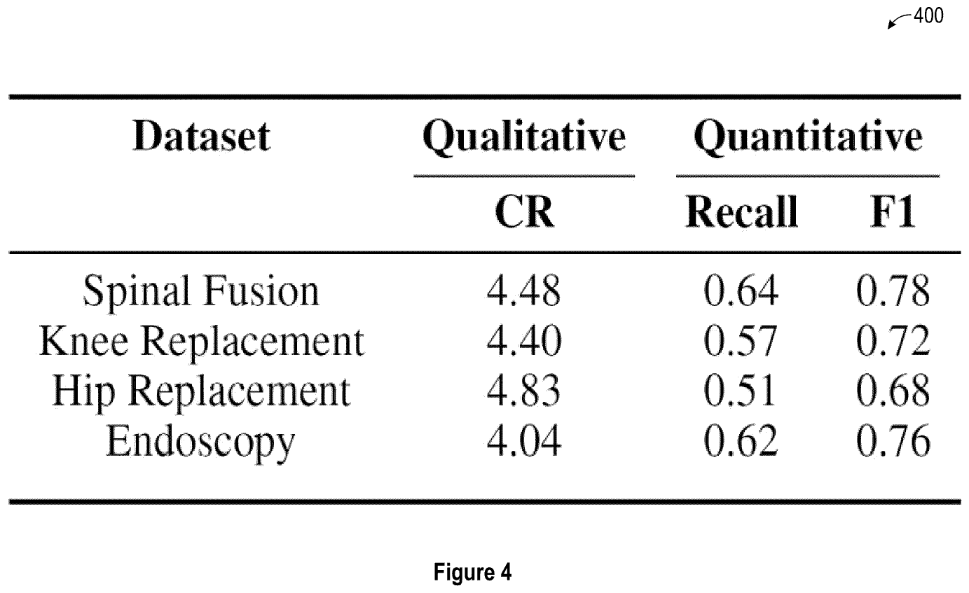

Qualitative Evaluation: The clinical relevance of MediClaimGPT's outputs was gauged by a panel of medical experts. The experts rated the outputs on a 1-5 scale, with 5 denoting high clinical relevance and 1 signifying low relevance despite potential accuracy. FIG. 4 shows a table 400 for evaluation of MediClaimGPT in zero-shot prediction for different datasets, according to some embodiments. The Clinical Relevance (CR) (averaged and shown in the table 300), suggest that the model's outputs were generally perceived as meaningful and relevant from a clinical perspective across all datasets.

Quantitative Evaluation: MediClaimGPT was quantitatively evaluated for its ability to correctly identify clinical events. As shown in the table 400, MediClaimGPT demonstrated varying degrees of recall and F1 scores across the datasets, with Spinal Fusion and Endoscopy showing relatively higher performance. The evaluation results underscore MediClaimGPT's efficacy in zero-shot clinical event prediction, with solid quantitative metrics and high qualitative ratings, especially in scenarios like Hip Replacement. This showcases the model's proficiency in a domain traditionally reliant on curated supervised datasets and significant domain expertise for feature engineering. MediClaimGPT's success in predicting clinical events without such datasets is a notable advancement. However, variability in performance across different conditions suggests the need for further refinement, particularly in enhancing recall in specific areas.

### Example Downstream Prediction

MediClaimGPT's performance was rigorously evaluated in downstream prediction tasks using diverse datasets Deval. The evaluation encompassed a range of representations and models, benchmarked against various baselines.

### Example Representations and Baselines

A baseline was established using a Bag-of-codes approach, where each patient is represented by the count of their medical codes. Because each medical code has an English description associated with it, pre-trained transformer-based language models, including BioBERT, Universal Sentence Encoder, and ADA-002, to convert medical codes into fixed-length representations. Additionally, a custom skip-gram based word2vec model was also trained on the claims corpus to represent medical codes.

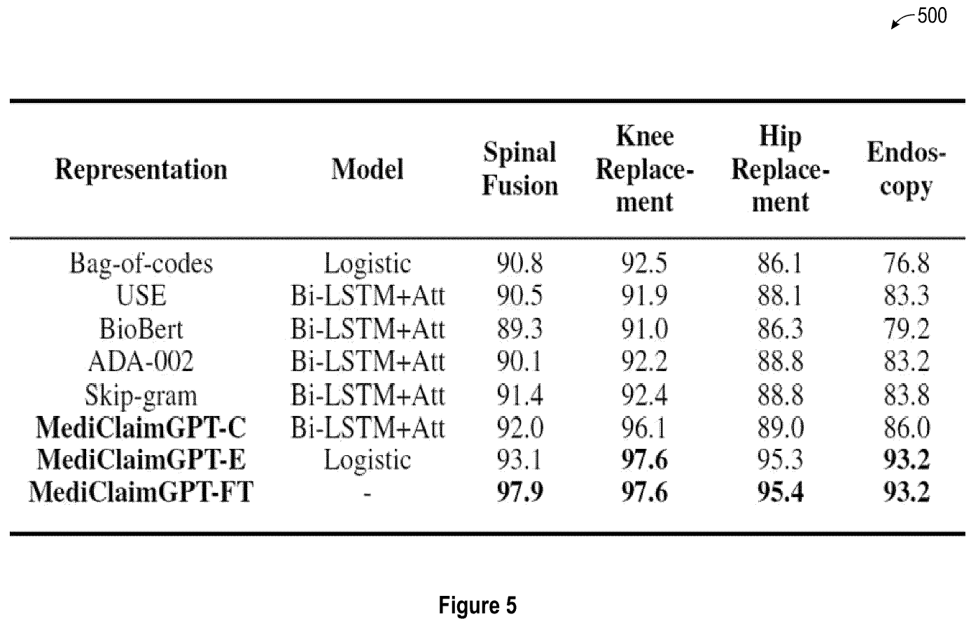

FIG. 5 shows a table 500 for classification performance (in ROC-AUC) across different representations and models for downstream prediction tasks, according to some embodiments. MediClaimGPT's embeddings were utilized in two distinct manners: (i) representing individual medical codes, and (ii) representing the entire patient claim sequence as fixed-length vectors, denoted as MediClaimGPT-C and MediClaimGPT-E respectively in the table shown in FIG. 5.

### Example Model Training and Evaluation

Models using logistic regression and Bi-LSTM with Attention (Bi-LSTM+Att) were trained with these representations. MediClaimGPT-FT represents the direct fine-tuning of MediClaimGPT for classification tasks. The Receiver Operating Characteristic Area Under the Curve (ROC-AUC) was employed as the performance metric.

### Example Experimental Setup for Downstream Prediction

Evaluation datasets was split in a 55%/25%/30% train/validation/test stratification. Training may be conducted over 100 epochs, with the best-performing models on the validation set saved after each epoch. The final performance was evaluated on the test set. Some embodiments used a batch size of 64, a learning rate α=10-5, and Adam optimizer with β1=0.9 and β2=0.999. Network weights were initialized using Xavier initialization, and L₂ regularization of 0.05 was applied, chosen based on grid search results from the validation set.

### Example Results

As illustrated in FIG. 5, MediClaimGPT's variants consistently surpassed other models in performance across various datasets. Notably, MediClaimGPT-E and MediClaimGPT-FT achieved the highest levels of classification accuracy. Although MediClaimGPT-C demonstrated commendable performance, its reliance solely on code-based embeddings limits its contextual understanding. These outcomes highlight the effectiveness of MediClaimGPT's embeddings (in MediClaimGPT-E) in capturing nuanced features and the model's enhanced capability through finetuning (in MediClaimGPT-FT). The standout performance of MediClaimGPT-FT particularly emphasizes the model's proficiency in direct classification tasks, confirming its potential as a versatile tool in healthcare data analysis.

### Example Synthetic Data Generation

To evaluate the utility of synthetic data (e.g., synthetic patient claims) generated by MediClaimGPT, the model was fine-tuned on the evaluation datasets, Deval. Special tokens, |pos| and |neg|, were introduced to enable the fine-tuned model to generate synthetic claims corresponding to positive and negative samples, respectively.

$${M}_{ft} = FineTune ( M , {eval}_{} , \vert pos \vert , \vert neg \vert ) \qquad ( 5 )$$

Where M_{ft} denotes the model after fine-tuning, utilizing |pos| or |neg| as prompts for generating the synthetic dataset. Example details on the experimental setup for fine-tuning and sample generation are provided below.

Example Fine-tuning. The hyperparameter settings from the unsupervised pretraining phase was largely retained, with the addition of a dropout rate of 0.5 and a learning rate of 6e-5. This configuration was found to be optimal, allowing the model to fine-tune effectively within just 5 epochs for all datasets. A linear learning rate decay schedule with a warmup over 0.5% of the training duration was also implemented.

Example Generation. 10,000 samples were generated for both positive and negative classes from each one of the fine-tuned models to create synthetic datasets. The generation parameters were set to a temperature of 0.3 and a maximum token limit of 500 per sample, optimizing for coherent and contextually relevant synthetic claims.

### Evaluation of Synthetic Datasets

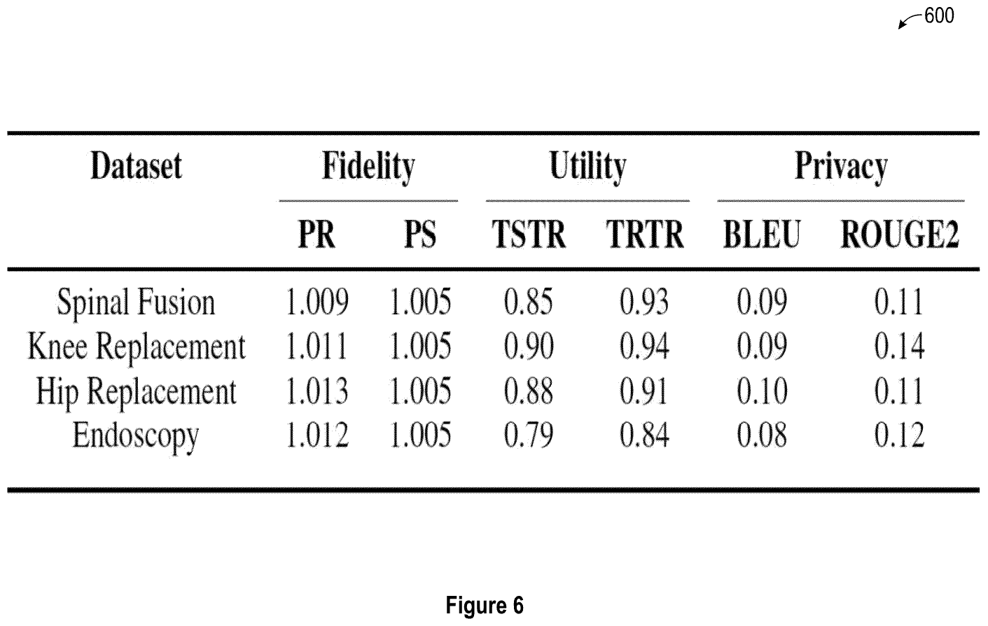

The evaluation framework for synthetic datasets prioritized fidelity, privacy and utility to ensure synthetic data quality and applicability. FIG. 6 shows a table 600 with results of evaluation of synthetic data, according to some embodiments. The table shows fidelity, utility and privacy results.

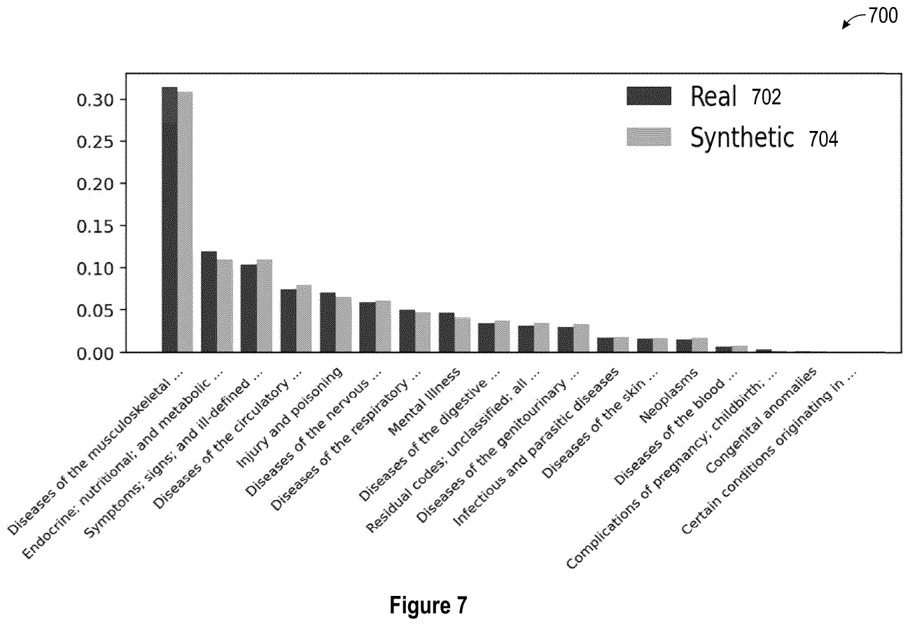

Fidelity: Fidelity assessment confirms the statistical resemblance of synthetic data to real data. It was assessed using perplexity and topic diversity. Perplexity (lower the better) is calculated on real and synthetic datasets (PR and PS). Given that PR and PS scores are close to each other and that PS scores are around 1.004-1.005 across all synthetic datasets-indicates a close alignment of the model's predictions with actual data distributions, implying high fidelity. Topic diversity was further analyzed using the Clinical Classification Software (CCS), mapping codes to higher-level categories. FIG. 7 shows a graph plot 700 for topic diversity between real and synthetic claims for Spinal Fusion dataset, according to some embodiments. The attributes of the real population 702 and the attributes of the synthetic population 704 show clinical similarity. As FIG. 7 shows, the significant overlap in CCS categories between real and synthetic datasets underscores the synthetic data's authentic representation of diverse clinical scenarios.

Utility: To evaluate utility, the Train-Synthetic-Test-Real (TSTR) and Train-Real-Test-Real (TRTR) approach was used, calculating ROC-AUC for both. The TSTR scores ranged from 0.79 to 0.90, while TRTR scores were slightly higher, ranging from 0.84 to 0.94. These results demonstrate that the synthetic data, although slightly less effective than real data, still holds significant utility for training models, particularly in scenarios where access to large volumes of real data may be limited.

Privacy: Privacy assessment ensures anonymity, by ensuring minimal overlap between real and synthetic datasets to minimize re-identification risks. BLEU and ROUGE2 metrics were used to evaluate this; BLEU measures the precision of the synthetic data against the real data, whereas ROUGE2 assesses recall. These metrics are crucial in this context because claims data inherently emphasizes the sequence of medical visits and specific diagnoses. Lower scores in these metrics indicate greater privacy, as they suggest less resemblance to real patient histories. The BLEU scores ranged from 0.08 to 0.10, and ROUGE2 scores from 0.11 to 0.14, confirming that the synthetic data maintains patient privacy by not closely mirroring any individual real patient's history. To summarize, the synthetic data generated by MediClaimGPT exhibits high fidelity and utility while effectively preserving privacy. This balance is crucial for creating synthetic datasets that are both functional for research and development purposes and preserve patient privacy.

### Example Training Dataset

In some embodiments, the training dataset, D, originates from an extensive administrative claims collection of a major U.S. healthcare insurer. Spanning six years, the dataset may cover diverse patient demographics and medical conditions, including over 70 million patients and 3 billion claims from various healthcare settings. The dataset comprises 92,000 unique diagnosis codes (ICD-10-CM) and 27,000 unique procedure codes (CPT). However, only approved claims may be included, resulting in a final count of 3 billion claims. Additionally, the dataset may be refined by excluding invalid codes, which often result from intake or ingestion errors, thereby narrowing it down to 85,000 diagnosis and 20,000 unique procedure codes.

### Experimental Setup for Zero-Shot Prediction

The temperature may be set to 0.7, balancing creativity and precision in the generated outcomes. Maximum tokens of 500 and a top-k sampling with k=100 may be used.

### Example System for Clinical Outcome Prediction

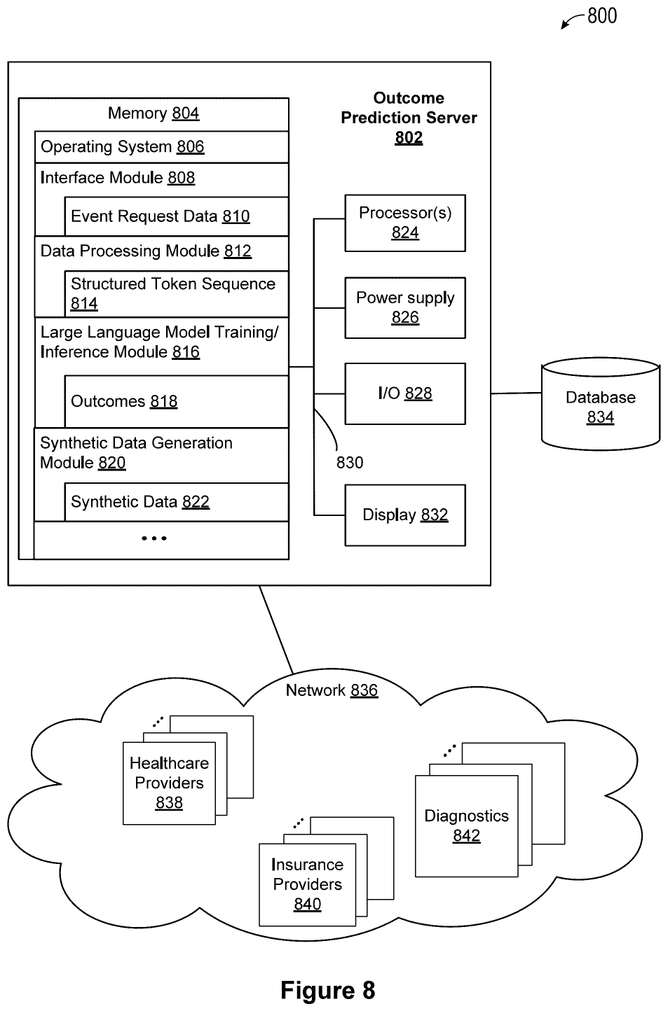

FIG. 8 is a system diagram of an example outcome prediction system 800, according to some embodiments. The system includes a server 802 typically includes one or more processor(s) 824, a memory 804, a power supply 826, an input/output (I/O) subsystem 828, and a communication bus 830 for interconnecting these components. Processor(s) 824 execute modules, programs and/or instructions stored in the memory 804 and thereby perform processing operations, including the methods described herein according to some embodiments. In some embodiments, the server 802 also includes a display 232 for displaying visualizations (e.g., outcomes, such as clinical outcomes, event requests data, such as claims data, probabilities). In some embodiments, the server 802 generates displays or visualizations, and transmits the visualization (e.g., as a visual specification) to a client device for display. Some embodiments of the server 802 include touch, selection, or other I/O mechanisms coupled to the server 802 via the I/O subsystem 828, to process input from users that select (or deselect) visual elements of a displayed visualization. Some aspects of the server 802 (e.g., the modules in the memory 804) are implemented in one or more client devices, according to some embodiments. In some embodiments, the client device (or software therein) processes user input and transmits a signal to the server 802 for processing.

In some embodiments, the memory 804 stores one or more programs (e.g., sets of instructions), and/or data structures, collectively referred to as “modules” herein. In some embodiments, the memory 804, or the non-transitory computer readable storage medium of the memory 804, stores the following programs, modules, and data structures, or a subset or superset thereof:
an operating system 806;
an interface module 808 that interfaces with data sources (e.g., providers) to monitor updates for and/or obtain event requests data 810 (e.g., claims data) from the data sources. Examples of claims data (sometimes referred to as healthcare claims data) are described above (e.g., in reference to FIG. 1), according to some embodiments;
a data processing module 812 processes and/or preprocesses the event requests data 810 to obtain structured token sequences 814. Examples of data processing/pre-processing, including tokenization, are described above, according to some embodiments;
a large language model training and/or inference module 816 uses the structured token sequences 814 to train and/or use large language model(s) to predict outcomes 818 (e.g., clinical outcomes); and/or
optionally, a synthetic data generation module 820 that uses a trained large language model (e.g., a model trained by the module 816) to generate synthetic data 822.

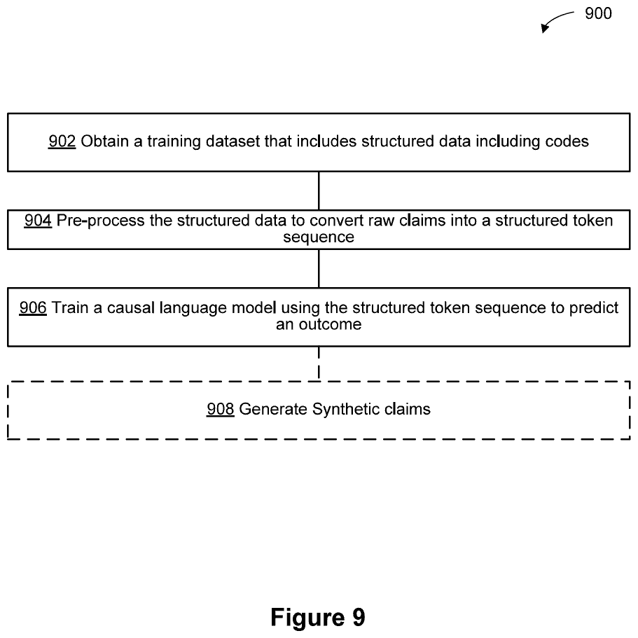

The above identified modules (e.g., data structures, and/or programs including sets of instructions) need not be implemented as separate software programs, procedures, or modules, and thus various subsets of these modules may be combined or otherwise re-arranged in various embodiments. In some embodiments, the memory 804 stores a subset of the modules identified above. In some embodiments, a database 834 (e.g., a local database and/or a remote database) stores one or more modules identified above and data associated with the modules. Furthermore, the memory 804 may store additional modules not described above. In some embodiments, the modules stored in the memory 804, or a non-transitory computer readable storage medium of the memory 804, provide instructions for implementing respective operations in the methods described below. In some embodiments, some or all of these modules may be implemented with specialized hardware circuits that subsume part or all of the module functionality. One or more of the above identified elements may be executed by the one or more of processor(s) 824. Operations of the module and use of the data in the memory 804 are further described below in reference to FIG. 9, according to some embodiments.

The I/O subsystem 828 communicatively couples the server 802 to one or more devices, such as devices corresponding to healthcare providers 838, insurance providers 840, and/or diagnostics 842 (e.g., providers of healthcare diagnostics, service providers predicting clinical outcomes), via a local and/or wide area communications network 836 (e.g., the Internet) via a wired and/or wireless connection. In some embodiments, the devices corresponding to the corresponding to healthcare providers 838, the insurance providers 840, and/or the diagnostics 842 push relevant information to the server 802. In some embodiments, the server 802 pulls relevant information from the devices corresponding to the healthcare providers 838, the insurance providers 840, and/or the diagnostics 842.

The communication bus 830 optionally includes circuitry (sometimes called a chipset) that interconnects and controls communications between system components.

### Example Method for Prediction of Clinical Outcomes

FIG. 9 is a flowchart of a method 900 for predicting outcomes using large language models, according to some embodiments. The method 9000 may be performed by a computing device (e.g., the server 802). The method may include obtaining (902) (e.g., by the interface module 808) a training dataset that includes structured data (e.g., the events requests data 810, sometimes referred to as claims data) including codes (e.g., medical codes). In some embodiments, the structured data includes a respective dataset for a plurality of individuals (e.g., patients), each dataset including a plurality of event requests (e.g., claims). Each individual may have a corresponding set of event requests, each event request may include a set of codes, and/or each code may be either a diagnosis code or a procedural code. In some embodiments, each event request in the structured data corresponds to an individual-provider encounter. Each event request may aggregate medical codes (e.g., diagnosis and/or procedure codes) in a non-sequential order.

In some embodiments, the training dataset includes event requests data (e.g., medical claims data) that covers a plurality of individual demographics and conditions (e.g., medical conditions) from a plurality of care settings (e.g., healthcare settings). In some embodiments, the training dataset includes billions of event requests corresponding to millions of individuals, tens of thousands of diagnosis codes, and tens of thousands of unique procedure codes. The training dataset may exclude invalid codes resulting from intake or ingestion errors.

The method may also include preprocessing (904) (e.g., by the data processing module 812) the structured data to convert raw event requests into a structured token sequence (e.g., the structured token sequence 814). In some embodiments, preprocessing the structured data includes performing a sorting algorithm to organize codes within each event request in the structured data into a clinically logical sequence, and may include chronologically ordering event requests for a respective individual (e.g., patient claims) to form a temporally sequenced dataset thereby enabling the causal language model to learn chronological order of events (e.g., medical events). In some embodiments, preprocessing the structured data includes inserting one or more delimiter tokens into the structured data for concatenating intra-event request codes, inter-event request codes for the respective individual (e.g., intra-claim codes, inter-claim codes for a patient), and data for different individuals (e.g., inter-patient data), thereby enabling batch data processing. In some embodiments, preprocessing the structured data includes tokenizing the structured data using a tokenizer to obtain a sequence of tokens. The tokenizer may preserve one or more delimiter tokens to maintain context of data (e.g., context of medical data). The tokenizer may be trained on event requests data (e.g., claims data) with a predetermined vocabulary size. In some embodiments, the tokenizer uses Byte-Level Byte-Pair Encoding for creating a fixed-size vocabulary balancing language specificity for a particular field (e.g., the medical field) with capacity of the causal language model. Some embodiments maintain a detailed and accurate representation of medical terms (specificity) while also managing the overall size and complexity (capacity) of the language model. In some embodiments, the tokenizer, which uses Byte-Level Byte-Pair Encoding, creates a vocabulary that includes specific medical terms for understanding and generation of medical language. Some embodiments keep the vocabulary size manageable so that the model remains efficient and performant.

The method may also include training (906) (e.g., by the large language model training or inference module 816) a causal language model using the structured token sequence to predict an outcome (e.g., the clinical outcome 818). In some embodiments, training the causal language model using the structured token sequence includes predicting a next code in the structured token sequence based on prior codes, thereby generating a sequence of codes for each event request in a causally coherent manner. In some embodiments, predicting the next code is modeled as a probability distribution over possible codes. The process of predicting the next code may include considering all possible codes and determining the probability of each one being the next correct code. This process creates a probability distribution, which reflects the likelihood of each potential code being selected as the next one. The model may then use this distribution to make an informed prediction about the next code. In some embodiments, the causal language model comprises a 12-layer transformer with 768-dimensional states across 12 attention heads, totaling about 125 million parameters. In some embodiments, the causal language model is trained on a 1,024-token context size to capture detailed individual histories (e.g., patient histories), using a batch size of 512. In some embodiments, the causal language model has a vocabulary size of 2,048 thereby optimizing handling of code hierarchies (e.g., medical code hierarchies) while maintaining computational efficiency. In some embodiments, the method further includes using zero-shot prompting for forecasting outcomes (e.g., patient health outcomes). In some embodiments, using zero-shot prompting includes inputting, to the causal language model, an individual's event request history (e.g., patient's claim history) for an observation period and analyzing output generated by the causal language model for event occurrence (e.g., clinical event occurrence). In some embodiments, temperature of the causal language model is set to 0.7, thereby balancing creativity and precision in generated outcomes, for zero-shot prediction. In some embodiments, a maximum token size of 500 and top-k sampling with k=100 are used for zero-shot prediction.

In some embodiments, the method 900 further includes generating (908) synthetic event requests (sometimes referred to as synthetic dataset; e.g., synthetic patient claims; using the trained large language model). For example, as described above, the trained large language model may be fine-tuned on an evaluation datasets, Deval. Special tokens (e.g., |pos| and |neg], described above) may be introduced to enable the fine-tuned model to generate synthetic event requests (e.g., synthetic claims) corresponding to positive and negative samples. Generating the synthetic dataset may include fine-tuning the trained causal language model that may include introducing special tokens |pos| and |neg| to enable the fine-tuned model to generate synthetic event requests corresponding to positive and negative samples, respectively:

$${M}_{ft} = FineTune ( M , {eval}_{} , \vert pos \vert , \vert neg \vert )$$

M denotes the trained causal language model, [symbol] ₑᵥₐₗ denotes the evaluation dataset, M_{ft} denotes the model after fine-tuning, and |pos| or |neg| are used prompts for generating the synthetic dataset. In some embodiments, hyperparameter settings from training the causal language model are retained during the fine-tuning with the addition of a dropout rate of 0.5 and a learning rate of 6e-5, to fine-tune within 5 epochs. In some embodiments, the fine-tuning uses a learning rate decay schedule with a warmup over 0.5% of training duration.

Although the description herein uses medical codes, patient claims data, patient health histories, and terms specific to the medical industry, such examples are used for illustrating the concepts and techniques described herein. The algorithms, processes and systems described herein may be used in any industry that uses similar terminologies (e.g., codes for automobile industry, airline industry) for predicting outcomes. As described above, MediClaimGPT, which is a large language model, effectively learned the practice of medicine when trained on a massive administrative claims dataset. The model's proficiency is showcased by the zero-shot prediction of clinical events and downstream classification tasks via various healthcare datasets. The model's application in creating synthetic claims data, holds tremendous promise for augmenting research and development, as demonstrated by strong evaluation results for fidelity, utility, and privacy. The proficiency of MediClaimGPT's embeddings described above, suggests that these embeddings can also be effectively utilized for analytical segmentation of patient populations and driving population health management strategy. Additionally, the generative capability of MediClaimGPT in forecasting medical events for patients could lead to new opportunities for digital twins. Some embodiments enrich MediClaimGPT by incorporating a wider range of medical codes, such as laboratory and drug codes, enhancing its medical understanding. Some embodiments integrate temporal information, like intervals between claims and episodic timeframes, to refine its predictive capabilities. These enhancements may lead to more personalized and efficient care, and expand the strategic application of LLMs in healthcare.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations were chosen and described in order to best explain the principles of the invention and its practical applications, to thereby enable others skilled in the art to best utilize the invention and various implementations with various modifications as are suited to the particular use contemplated.

What is claimed is:

1. A method of training a causal language model for predicting outcomes, the method comprising:
obtaining a training dataset that includes structured data including codes;
preprocessing the structured data to convert raw event requests into a structured token sequence, including:
performing a sorting algorithm to organize codes within each event request in the structured data into a clinically logical sequence, including chronologically ordering event requests for a respective individual to form a temporally sequenced dataset thereby enabling a machine learning model to learn chronological order of events;
inserting one or more delimiter tokens into the structured data for concatenating intra-event request codes, inter-event request codes for the respective individual, and data for different individuals, thereby enabling batch data processing; and
tokenizing the structured data using a tokenizer to obtain a sequence of tokens, wherein the tokenizer preserves the one or more delimiter tokens to maintain context of event request data, wherein the tokenizer is trained on event request data with a predetermined vocabulary size; and
training a causal language model using the structured token sequence to predict an outcome, wherein training the causal language model comprises predicting a next code in the structured token sequence based on prior codes, thereby generating a sequence of codes for each event request in a causally coherent manner, wherein predicting the next code is modeled as a probability distribution over possible codes.

2. A method of training a causal language model for predicting clinical outcomes, the method comprising:
obtaining a training dataset that includes structured data including codes;
preprocessing the structured data to convert raw event requests into a structured token sequence; and
training a causal language model using the structured token sequence to predict an outcome.

3. The method of claim 2, wherein the structured data includes a respective dataset for a plurality of individuals, each dataset including a plurality of event requests, wherein each individual has a corresponding set of event requests, each event request comprising a set of codes, wherein each code is either a diagnosis code or a procedural code.

4. The method of claim 2, wherein each event request in the structured data corresponds to an individual-provider encounter, aggregates medical codes in a non-sequential order.

5. The method of claim 2, wherein preprocessing the structured data comprises:
performing a sorting algorithm to organize codes within each event request in the structured data into a clinically logical sequence, including chronologically ordering event requests for a respective individual to form a temporally sequenced dataset thereby enabling a machine learning model to learn chronological order of events.

6. The method of claim 5, wherein the sorting algorithm σ organizes the codes within each event request cᵢⱼ into a clinically logical sequence,
$${c}_{i j}^{l} = σ ( {e}_{ijl} , {e}_{ij 2} , … , {e}_{ijk} ) ,$$
wherein event requests
$${C}_{i}^{l} = {c}_{i 1}^{l} , {c}_{i 2}^{l} , … , {c}_{i C}^{l}$$
is chronologically ordered as
$${′}^{} = {⋃}_{p = 1}^{P} \{ sort ( {C}_{p} , date ) \}$$
forming a temporally sequenced dataset, enabling the causal language model to learn the chronological order of events.

7. The method of claim 2, wherein preprocessing the structured data comprises:
intra-event request codes, inter-event request codes for the respective individual, and data for different individuals, thereby enabling batch data processing.

8. The method of claim 2, wherein preprocessing the structured data comprises:
tokenizing the structured data using a tokenizer to obtain a sequence of tokens, wherein the tokenizer preserves one or more delimiter tokens to maintain context of data, wherein the tokenizer is trained on event requests data with a predetermined vocabulary size.

9. The method of claim 8, wherein the tokenizer uses Byte-Level Byte-Pair Encoding for creating a fixed-size vocabulary balancing medical language specificity with capacity of the causal language model.

10. The method of claim 8, wherein the causal language model is trained on the sequence of tokens to predict a subsequent token in the sequence, with a loss function measuring the accuracy of predictions
$$Loss ( Θ ) = - {\sum }_{t = 1}^{L} log P ( t \vert t - 1 , t - 2 , … , 1 ; Θ ) , wherein$$
$$P ( t \vert t - 1 , t - 2 , … , 1 ; Θ )$$
represents the causal language model's assigned probability to a true next token t, given all previous tokens in the sequence.

11. The method of claim 2, wherein the training dataset includes event requests data that covers a plurality of individual demographics and conditions from a plurality of care settings.

12. The method of claim 2, wherein the training dataset comprises billions of event requests corresponding to millions of individuals, tens of thousands of diagnosis codes, and tens of thousands of unique procedure codes, and wherein the training dataset excludes invalid codes resulting from intake or ingestion errors.

13. The method of claim 2, wherein training the causal language model using the structured token sequence comprises predicting a next code in the structured token sequence based on prior codes, thereby generating a sequence of codes for each claim in a causally coherent manner.

14. The method of claim 13, wherein predicting the next code is modeled as a probability distribution over possible codes.

15. The method of claim 14, wherein the probability distribution over the possible codes is formulated as P (eᵢⱼₖ euj; 0)=M (eu), wherein 0 denotes the parameters of the causal language model, wherein sequence of codes eᵢⱼ=(eijt, eᵢⱼ₂, eᵢⱼ (k-1)) for the j^{th} event request of the i^{th} individual, wherein the language model predicts the next code eᵢⱼₖ thereby generating the sequence of codes for each event request in a causally coherent manner, reflective of the actual progression of events documented in the event requests data.

16. The method of claim 2, further comprising:
using zero-shot prompting for forecasting outcomes.

17. The method of claim 16, wherein using zero-shot prompting comprises inputting, to the causal language model, an individual's event request history for an observation period and analyzing output generated by the causal language model for event occurrence.

18. The method of claim 2, further comprising:
generating a synthetic dataset based on fine-tuning the trained causal language model on an evaluation dataset.

19. The method of claim 18, wherein fine-tuning the trained causal language model comprises introducing special tokens |pos| and |neg| to enable the fine-tuned model to generate synthetic event requests corresponding to positive and negative samples, respectively
$${M}_{ft} = FineTune ( M , {eval}_{} , \vert pos \vert , \vert neg \vert )$$
wherein M denotes the trained causal language model, [symbol] eval denotes the evaluation dataset, M_{ft} denotes the model after fine-tuning, and |pos| or |neg| are used prompts for generating the synthetic dataset.

20. A computer system for predicting outcomes using large language models, comprising:
one or more processors;
a display; and
memory;
wherein the memory stores one or more programs configured for execution by the one or more processors, and the one or more programs comprising instructions for:
obtaining a training dataset that includes structured data including codes;
preprocessing the structured data to convert raw event requests into a structured token sequence, including:
performing a sorting algorithm to organize codes within each event request in the structured data into a clinically logical sequence, including chronologically ordering event requests for a respective individual to form a temporally sequenced dataset thereby enabling a machine learning model to learn chronological order of events;
inserting one or more delimiter tokens into the structured data for concatenating intra-event request codes, inter-event request codes for the respective individual, and data for different individuals, thereby enabling batch data processing; and
tokenizing the structured data using a tokenizer to obtain a sequence of tokens, wherein the tokenizer preserves the one or more delimiter tokens to maintain context of event request data, wherein the tokenizer is trained on event request data with a predetermined vocabulary size; and
training a causal language model using the structured token sequence to predict an outcome, wherein training the causal language model comprises predicting a next code in the structured token sequence based on prior codes, thereby generating a sequence of codes for each event request in a causally coherent manner, wherein predicting the next code is modeled as a probability distribution over possible codes.
